(19) United States
(12) Patent Application Publication
(10) Pub. No.: US 2026/0314591 A1
(43) Pub. Date: Oct. 15, 2026

(54) MULTI-LAYER GRAFT

(71) Applicant: Tiger Wound Care Medical, LLC, Conshohocken, PA (US)

(72) Inventors: Oliver BURCKHARDT, Philadelphia, PA (US); Joanna MARQUEZ, San Antonio, TX (US); Raymond Anthony GOMEZ, San Antonio, TX (US)

(21) Appl. No.: 19/467,528

(22) Filed: Feb. 2, 2026

Related U.S. Application Data
(60) Provisional application No. 63/752,027, filed on Jan. 31, 2025.

(51) Int. Cl. A01N 1/128

(52) U.S. Cl. CPC A01N 1/128

(57) ABSTRACT

The present disclosure describes preserving and/or adding layers to natural tissue to yield a multi-layer graft with a rich composition of an extracellular matrix, potentially supporting a stronger structural barrier for clinical applications such as wound care. Processes for minimally manipulating the placental tissue to retain the intermediate layer with the corresponding proteins and growth factors are described. Processes for incorporating a second amnion that may improve handling by adding durability and thickness are also described. The second amnion may further increase the amount and/or concentration of components native to the placenta membrane. The multi-layer grafts provide biological complexity to enhance natural healing processes.

### CROSS-REFERENCE TO RELATED APPLICATIONS

This application claims the benefit of priority to U.S. Provisional Application No. 63/752,027, filed on Jan. 31, 2025, the entire contents of which are incorporated by reference herein.

### BACKGROUND

Placental tissue forms a placental membrane 10 of an amniotic sac 12, as illustrated in FIG. 1. The amniotic sac 12 contains amniotic fluid and serves as a protective barrier for a developing fetus. The placental membrane 10 shields the fetus from external environmental factors and potential infections. The placental membrane 10 has a high concentration of extracellular matrix (ECM) and associated components that provide structural integrity and a supportive biological environment for the fetus to grow and develop. The placental membrane 10 further includes a placental globe (or disc or crown) 14 that separates a placenta 16 from the amniotic fluid. The placental globe 14 attaches to an umbilical cord 18 that provides oxygen and nutrients to the fetus from the mother via the placenta 16. The remaining portion 20 of the placental membrane 10 (excluding the placental globe 14) extends around the amniotic fluid to form the amniotic sac 12.

The tissue of the placental membrane 10 is composed of three main layers—an amnion 102, an intermediate layer 104, and a chorion 106. Each of the layers 102, 104, 106 contributes distinct structural and biochemical properties.

The amnion 102 is the innermost layer closest to the fetus. The amnion 102 is a thin, tough, and nearly transparent membrane that forms the amniotic sac 12 surrounding and protecting the developing fetus. As illustrated in FIG. 1B, the amnion 102 is composed of an epithelial layer, a basement membrane, a compact layer, and a fibroblast layer. The amnion 102 has ECM that is particularly rich in collagens I and III and other matrix-associated proteins. The epithelial layer may form an epithelial side forming the outer boundary enclosing the amniotic fluid, and the fibroblast layer may form a stromal side contacting the intermediate layer 104.

The intermediate layer 104 is a distinct acellular spongy layer between the amnion 102 and chorion 106. The intermediate layer 104 is rich in proteoglycans, glycoproteins, hyaluronic acid (HA), and collagen type III. The intermediate layer 104 serves as a reservoir for additional ECM components. The intermediate layer 104 also contains naturally occurring growth factors such as EGF (epidermal growth factor), VEGF (vascular endothelial growth factor), PDGF-AA (platelet-derived growth factor), and ANG-2 (angiopoietin-2). These growth factors of the intermediate layer 104 play essential roles in fetal development and participate in cellular processes such as vascularization, proliferation, and tissue remodeling.

The chorion 106 is the outermost layer of the placental tissue. The chorion 106 is a thicker, more opaque membrane than the amnion 102. The chorion 106 contains the reticular layer, pseudo-basement membrane, and trophoblast layer. The chorion 106 has ECM composed of collagens I-VI, laminin, elastin, along with other structural components. The trophoblast layer forms a maternal side of the placental membrane 10.

### SUMMARY

Placental tissue is naturally enriched with various ECM components and growth factors. Incorporating distinct layers may optimize their composition by providing varying amounts of structural and non-structural proteins which may affect the allograft's suitability and efficacy as a physical barrier. For example, collagen, elastin, proteoglycans, HA, and growth factors play vital roles in the structural and biochemical properties of placental tissue. Among these structural proteins, collagen is essential for forming a protective barrier, while elastin and proteoglycans also support this function. The intermediate layer is especially rich in a number of proteins, including collagen, hyaluronic acid, and growth factors. Due to the susceptibility of the separation of the amnion 102 and chorion 106, processing the placental tissue increases susceptibility of separation and loss of the intermediate layer 104.

Embodiments of the present disclosure preserve and/or add layers to natural tissue to yield a multi-layer graft with a rich composition of ECM, potentially supporting a stronger structural barrier for clinical applications such as wound care. For example, the present disclosure provides processes of minimally manipulating the placental tissue to retain the intermediate layer with the corresponding proteins and growth factors. The present disclosure further provides processes of incorporating a second amnion that may improve handling by adding strength and/or thickness. The second amnion may further increase the amount and/or concentration of proteins. The multi-layer grafts of the present disclosure provide biological complexity to enhance natural healing processes.

A first aspect of the present disclosure is directed to a multi-layer graft. The multi-layer graft may include a first amnion having a first stromal side and a first epithelial side; a chorion; and a second amnion having a second stromal side and a second epithelial side, wherein the chorion is positioned between the first stromal side and the second stromal side, and the first epithelial side and the second epithelial side are exposed.

In some embodiments, the multi-layer graft may include one or more of the following features. The multi-layer graft may include an intermediate layer between the first amnion and the chorion. The first amnion, the intermediate layer, and the chorion may be derived from a full thickness of a placental tissue. The first amnion may be at least partially derived from a first portion of a placental membrane, and the second amnion may be at least partially derived from a second portion of the placental membrane. The second portion may be a placental globe, and the first portion may be a portion of the placental membrane excluding the placental globe. The second amnion may be in direct contact with the chorion. The first epithelial side of the first amnion may have a first layer of epithelial cells, and the second epithelial side of the second amnion may have a second layer of epithelial cells. The multi-layer graft may have a thickness of at least about 100 microns. The chorion may have a thickness constituting from about 38% to about 81%, inclusive, of a total thickness of the multi-layer graft. The thickness of the chorion may constitute about 60% to about 75%, inclusive, of the total thickness of the multi-layer graft. The first amnion and the second amnion may have a combined thickness constituting from about 9% to about 29%, inclusive, of a total thickness of the multi-layer graft. The combined thickness of the first amnion and the second amnion may constitute from about 12% to about 22%, inclusive, of the total thickness of the multi-layer graft. The intermediate layer may have a thickness constituting from about 7% to about 36%, inclusive, of a total thickness of the multi-layer graft. The thickness of the intermediate layer may constitute from about 8% to about 18%, inclusive, of the total thickness of the multi-layer graft. The multi-layer graft may have collagen in a concentration of from about 500 μg/cm² to about 1750 μg/cm², inclusive. The multi-layer graft may have elastin in a concentration of from about 7 mg/cm² to about 14 mg/cm², inclusive. The multi-layer graft may have proteoglycans in a concentration of from about 750 μg/cm² to about 3250 μg/cm², inclusive. The multi-layer graft may have hyaluronic acid in a concentration of from about 25 μg/cm² to about 175 μg/cm², inclusive. The first amnion, the chorion, and the second amnion may be adhered together.

A second aspect of the present disclosure is directed to a method. The method may include receiving a first amnion and a chorion derived from a first placental tissue; receiving a second amnion derived from a second placental tissue; and applying a stromal side of the second amnion to a maternal side of the chorion to form a multi-layer graft with a first epithelial side of the first amnion and a second epithelial side of the second amnion exposed.

In some embodiments, the method may include one or more of the following features. The method may include receiving an intermediate layer from the first placental tissue. The first amnion, the intermediate layer, and the chorion may be derived from a full thickness of the first placental tissue. The first placental tissue may be at least partially derived from a first portion of a placental membrane, and the second placental tissue may be at least partially derived from a second portion of the placental membrane. The second portion may be a placental globe, and the first portion may be a portion of the placental membrane excluding the placental globe. The method may further include separating an amnion portion from the first amnion, based on the second amnion insufficiently covering the maternal side of the chorion; and applying a stromal side of the amnion portion to the maternal side of the chorion. The method may further include separating a chorion portion from the chorion; and discarding the chorion portion.

### BRIEF DESCRIPTION OF THE DRAWINGS

In order that the present disclosure may be readily understood, aspects of the invention are illustrated by the way of examples in the accompanying drawings, in which like parts are referred to with like reference numeral throughout.

FIG. 1A illustrates anatomy of an embryo.

FIG. 1B illustrates compositions of a placental membrane.

FIGS. 2A-C illustrate histological compositions of exemplary embodiments of multi-layer grafts.

FIG. 3 is a stacked column graph illustrating the compositional analysis of the multi-layer grafts of FIGS. 2A-C based on proportional thickness.

FIG. 4 is a bar graph illustrating the mean weight normalized to surface area of the multi-layer grafts of FIGS. 2A-C.

FIG. 5A-C are images of the exemplary multi-layer grafts of FIGS. 2A-C.

FIG. 6A is a bar graph illustrating the total protein concentrations normalized to surface area of the multi-layer grafts of FIGS. 2A-C.

FIG. 6B is a bar graph illustrating the total protein weights normalized to a weight of the multi-layer grafts of FIG. 2A.

FIGS. 7A-C are images of distribution of collagen in the multi-layer grafts of FIG. 2A-C.

FIG. 7D is a bar graph illustrating the amounts of total collagen normalized by a surface area of the multi-layer grafts of FIG. 2A-C.

FIG. 8A is a bar graph illustrating the amounts of elastin normalized by the surface area of the multi-layer grafts of FIG. 2A-C.

FIG. 8B is a bar graph illustrating the amounts of proteoglycans normalized by the surface area of the multi-layer grafts of FIGS. 2A-C.

FIG. 8C is a bar graph illustrating the amounts of hyaluronic acid normalized by the surface area of the multi-layer grafts of FIGS. 2A-C.

FIG. 9A is a bar graph illustrating the amounts of PDGF-AA normalized by the surface area of the multi-layer grafts of FIGS. 2A-C.

FIG. 9B is a bar graph illustrating the amounts of EGF normalized by the surface area of the multi-layer grafts of FIGS. 2A-C.

FIG. 9C is a bar graph illustrating the amounts of VEGF normalized by the surface area of the multi-layer grafts of FIGS. 2A-C.

FIG. 9D is a bar graph illustrating the amounts of ANG-2 normalized by the surface area of the multi-layer grafts of FIGS. 2A-C.

FIG. 10 is a flow chart for a method of producing embodiments of the multi-layer graft of FIGS. 2A-C.

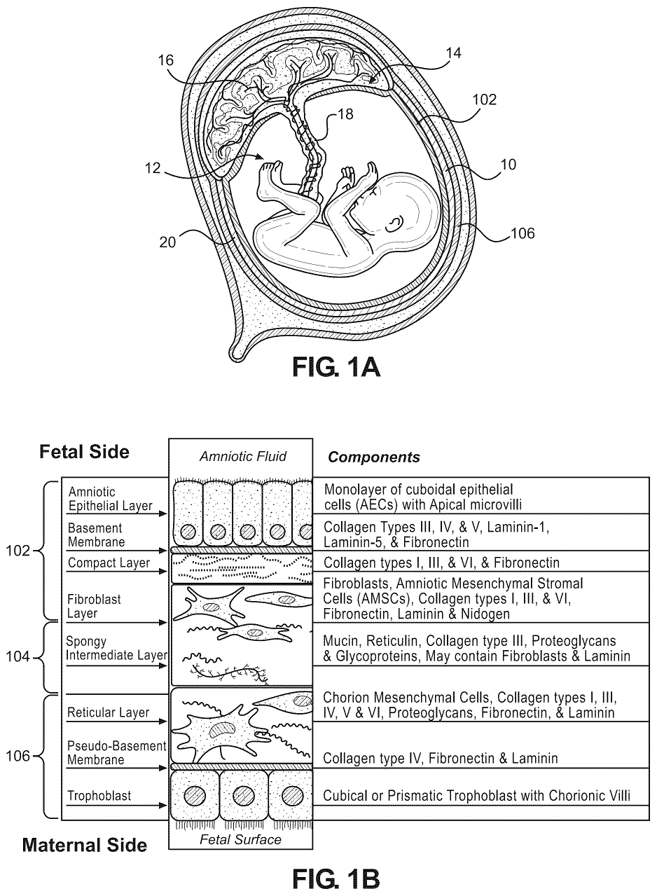

### DETAILED DESCRIPTION

The following description of the embodiments is merely exemplary in nature and is in no way intended to limit the subject matter of the present disclosure, their application, or uses.

As used throughout, ranges are used as shorthand for describing each and every value that is within the range. Any value within the range can be selected as the terminus of the range.

For the purposes of this specification, unless otherwise indicated, all numbers expressing quantities, percentages or proportions, and other numerical values used in the specification, are to be understood as being modifiable in all instances by the term “about.” The use of the term “about” may be applied to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent, alternatively ±5 percent, alternatively ±1 percent, alternatively ±0.5 percent, and alternatively ±0.1 percent of the given numeric value provided such a deviation does not alter the end function or result of the value. Accordingly, unless indicated to the contrary, the numerical parameters set forth in this specification include a disclosure of approximations that can vary depending upon the desired properties sought to be obtained by the present invention.

It is noted that, as used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items. For example, as used in this specification and the following claims, the terms “comprise” (as well as forms, derivatives, or variations thereof, such as “comprising” and “comprises”), “include” (as well as forms, derivatives, or variations thereof, such as “including” and “includes”) and “has” (as well as forms, derivatives, or variations thereof, such as “having” and “have”) are inclusive (i.e., open-ended) and do not exclude additional elements or steps. Accordingly, these terms are intended to not only cover the recited element(s) or step(s), but may also include other elements or steps not expressly recited. Furthermore, as used herein, the use of the terms “a” or “an” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The present invention relates to a multi-layer graft configured to treat patients. The multi-layer graft herein may replicate placental tissue and retain the native ECM and proteins of the donor tissue. Further, the multi-layer graft herein may retain structural components such as collagen, elastin, hyaluronic acid and proteoglycans. The retained cellular components may maintain the structural integrity and improve the biological function of the multi-layer graft.

The present disclosure provides embodiments of multi-layer placental grafts having the different layers of composition. In some embodiments, the multi-layer graft comprises at least two layers. In some instances, the multi-layer graft comprises at least three layers, alternatively at least four layers, and alternatively at least five layers. In some instances, the multi-layer graft comprises at least four layers. In some instances, the multi-layer graft comprises layers of placental tissue. Given that the placental layers are naturally enriched with different ECM components and growth factors, multi-layer grafts incorporating distinct layers may improve overall composition from natural placental tissue. For example, processed placental grafts contain different or varying amounts of structural and non-structural proteins which may affect the graft's suitability and efficacy as a physical barrier. Among these structural proteins, collagen is essential for forming a protective barrier, while elastin and proteoglycans also support this function. The retention of ECM constituents and growth factors in these materials reflects the biological complexity to the final composition of placental-derived grafts.

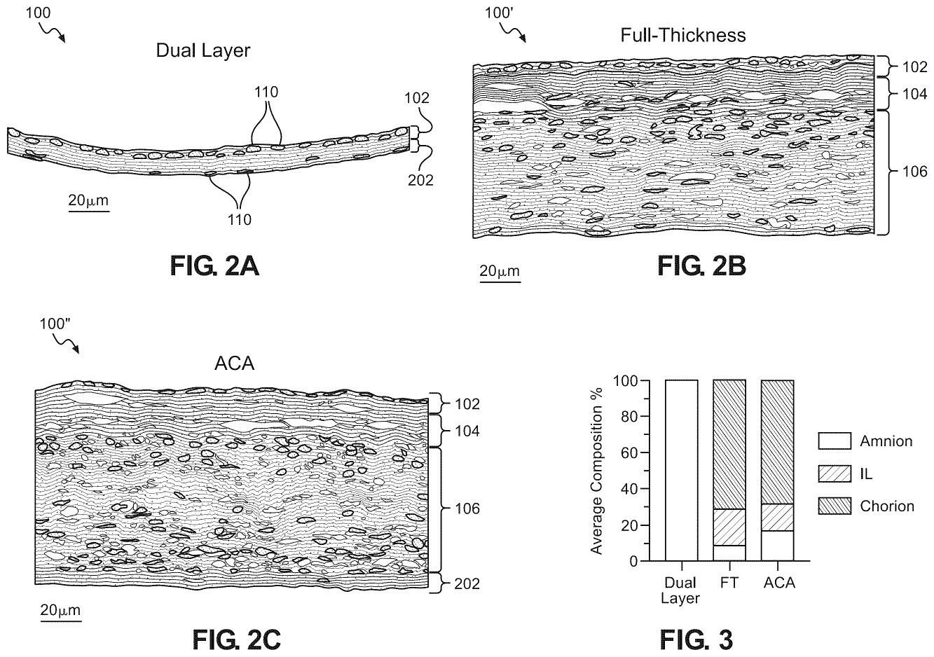

FIGS. 2A-C illustrate compositional analysis of embodiments of a multi-layer graft, including histological analysis based on H&E staining. The staining confirms the integrity of the layers and highlights structural differences among the multi-layer grafts

FIG. 2A illustrates a first embodiment of the multi-layer graft comprising at least two layers or a dual layer graft 100. The dual layer graft 100 may comprise a first amnion 102 and a second amnion 202 in a back-to-back manner. In some embodiments, the dual layer graft 100 may be formed by folding amnion from a single placental tissue back on to itself. The placental tissue of the dual layer graft 100 may be extracted from the placental globe 14 of the placental tissue due the natural separation of the amnion 102 and the chorion 104. This may simplify the process by removing any need to bluntly dissect the layers of the placental tissue. In some embodiments, the dual layer graft 100 may be formed by pressing amnion from two separate portions of placental tissue back-to-back.

In some embodiments, the portions of placental tissue may be extracted from different portions of the placenta. For example, in some embodiments, the first amnion 102 may be at least partially (e.g., entirely) derived from the portion 20 of the membrane 10 excluding the placental globe 14. In some embodiments, the second amnion 202 may be at least partially (e.g., entirely) derived from the placental globe 14. In some embodiments, the first amnion 102 and second amnion 202 may both be extracted from the placental globe 14. In some embodiments, the first amnion 102 and the second amnion 202 may both be extracted from the portion 20 of the membrane 10 excluding the placental globe 14. The first amnion 102 may be extracted from the placental globe 14, and the second amnion 202 may be extracted from a portion 20 of the membrane excluding the placental globe 14. Thus, at least one of the first amnion 102 and the second amnion 202 may be prepared from placental tissue by separating and discarding the chorion from the amnion 102, 202. In some embodiments, the intermediate layer may be removed (intentionally or unintentionally) from one or both of the first amnion 102 and the second amnion 202 before forming the dual layer graft 100.

Each of the first amnion 102 and the second amnion 202 may have a stromal side and an epithelial side. The dual layer graft 100 may be formed back-to-back with the stromal side of the first amnion 102 facing the stromal side of the second amnion 202, exposing the epithelial side of the first amnion 102 and the epithelial side of the second amnion 202. At least a portion of the intermediate layer 104 may be retained on one or both of the amnions 102, 202. The adhesion may be at least partially formed by the sticky layer of the intermediate layer 104. Additionally or alternatively, the adhesion may be formed by drying the dual layer graft 100, as discussed herein. Additionally or alternatively, at least a portion of the intermediate layer may be removed from one or both of the amnion 102, 202. In some embodiments, removal of the intermediate layer may expose the fibroblast layer of the respective amnion 102, 202. The fibroblast layer of the first amnion 102 may at least partially adhere with the fibroblast layer of the second amnion 202, for example by intertwining or mating when pressed against each other. Thus, the amnion 102 may adhere to the second amnion 202 at least partially through direct contact.

Exposure of the epithelial layers of each of the amnion 102, 202 may provide a tissue adhesion barrier. For example, the dual layer graft 100 may be implanted as a patch to prevent unwanted adhesion of tissue in orthopedic, fetal, wound, ophthalmic, neural and/or nervous system applications. As further illustrated in FIG. 2A, at least one (or both) of the epithelial sides of the amnion 102, 202 may be populated with epithelial cells 110 preserved from the native tissue. The epithelial cells 110 may be viable or non-viable. In some embodiments, at least one of the epithelial sides of the amnion 102, 202 may be scraped removing cells, excess tissue and/or cellular debris.

FIG. 2B illustrates a second embodiment of the multi-layer graft comprising three layers or a full-thickness graft 100′. As illustrated, the full-thickness graft 100′ may comprise an amnion 102, an intermediate layer 104, and a chorion 106. The full-thickness graft 100′ may be prepared by minimally manipulating or gently washing the placental membrane 10 to ensure that at least a portion of the intermediate layer 104 is retained in the full-thickness graft 100′. The intermediate layer 104 may contribute hyaluronic acid and growth factors that may be beneficial for several uses. The full-thickness graft 100′ may be further dried to retain the structural integrity of the full-thickness graft 100′. Extraction of the placental membrane 10 may cause at least partial separation of the amnion 102 and the chorion 106. Thus, the full-thickness graft 100′ may be reconstructed by applying and/or extending the amnion 102 over the chorion 106 (e.g., with at least part of the intermediate layer 104 therebetween) to create a smooth surface of the placental tissue. The intermediate layer 104 may be used to adhere the amnion 102 and the chorion 104. Additionally or alternatively, the adhesion may be formed by drying the dual layer graft 100, as discussed herein.

FIG. 2C illustrates a third embodiment of the multi-layer graft comprising at least four layers or an amnion-chorion-amnion (ACA) graft 100″. As illustrated, the ACA graft 100″ may comprise a first amnion 102, an intermediate layer 104, a chorion 106, and a second amnion 202. The ACA graft 100″ may incorporate the full-thickness graft 100′ of FIG. 2B for the first amnion 102, the intermediate layer 104, the chorion 106. The ACA graft 100″ may incorporate the second amnion 202, similar to the dual layer graft 100 of FIG. 2A. As further discussed with reference to the dual layer graft 100, at least one (or both) of the epithelial sides of the amnion 102, 202 may be populated with epithelial cells 110 preserved from the native tissue. Accordingly the entire disclosure of the dual layer graft 100 and/or the full-thickness graft 100′ is incorporated herein for sake of brevity, unless otherwise indicated.

Each of the first amnion 102, the intermediate layer 104, the chorion 106, and second amnion 202 may be prepared from the placental membrane 10 from a single donor. The first amnion 102, first intermediate layer 104, chorion 106 may be formed from the portion 20 of the placental membrane 10 excluding the placental globe 14, where the amnion 102 and the chorion 106 are naturally laminated/adhered together, usually by the intermediate layer. In some embodiments, at least a portion (or the entirety) of the second amnion 202 may be derived from the placental globe 14 of the placental membrane 10, where natural separation may occur between the amnion and the chorion. Deriving at least a portion (or the entirety) of the second amnion 202 from the placental globe 14 may facilitate handling by reducing steps. However, at least a portion (or the entirety) of the second amnion 202 may be formed from the portion 20 of the placental membrane 10 excluding the placental globe 14, for example when the placental globe 14 is not available to sufficiently cover the maternal side of the chorion 106.

In some embodiments, at least a portion of the intermediate layer remains on the stromal side of the second amnion 202. In some embodiments, at least portion of the intermediate layer may be removed from the stromal side of the second amnion 202. For example, the stromal side of the second amnion 202 may be scraped to remove at least a portion of the intermediate layer.

As illustrated in FIG. 2C, the ACA graft 100″ may be formed by positioning the chorion 106 between the first amnion 102 and the second amnion 202, such that the epithelial side of the first amnion 102 and the epithelial side of the second amnion 202 are exposed. As discussed with reference to the dual layer graft 100, at least a portion of the intermediate layer may be retained on the second amnion 202. The adhesion may be at least partially formed by the sticky layer of the intermediate layer. Additionally or alternatively, the adhesion may be formed by drying the ACA graft 100″, as discussed herein. For example, drying the intermediate layer 104 (between the amnion 102 and the chorion 104) and the intermediate layer (between the chorion and the second amnion 202) may bond or fuse the layers of the ACA graft 100″ together. Additionally or alternatively, at least a portion of the intermediate layer may be removed from the second amnion 202. In some embodiments, removal of the intermediate layer may expose the fibroblast layer of the second amnion 202. The fibroblast layer of the second amnion 202 may at least partially adhere with the chorion 106, for example by intertwining or mating when pressed against each other.

The ACA graft 100″ may provide the collagen-rich structure of the full-thickness graft 100′ with improved stiffness for handling and biodegradation with the layering of the second amnion 202. Table 1 illustrates exemplary thicknesses and compositions of the multi-layer grafts.

TABLE 1

| | Dual Layer | Full-Thickness | ACA |
|---|---|---|---|
| Total | 26.97 ± 16.89 | 133.23 ± 77.77 | 153.19 ± 57.74 |
| Thickness (μm)^{a} | | | |
| Amnion (%)^{b} | 100% | 4-12% | 9-29% |
| Intermediate | | 11-33% | 7-36% |
| Layer %)^{b} | | | |
| Chorion (%)^{b} | | 56-86% | 38-81% |

^{a}Values are represented in mean ± standard deviation (SD), measured in micrometers (μm).
^{b}Values are represented in a minimum-a maximum percentage of each layer per allograft.

As illustrated in Table 1, the second amnion 202 may increase the thickness of the ACA graft 100″ relative to the full-thickness graft 100′. The full-thickness graft 100′ and the ACA graft 100″ may be significantly thicker than the dual layer graft 100. The full-thickness graft 100′ may have an overall mean thickness greater than the dual layer graft 100 due to the retention of the intermediate layer 104 and the chorion 106. The ACA graft 100″ may have an overall mean thickness greater than the full-thickness graft 100′ due to the layering of the second amnion 202. Thus, the overall mean thickness of the full-thickness graft 100′ may be at least about 4 times greater than the dual layer graft 100, such as about 5 times greater. The overall mean thickness of the ACA graft 100″ may be at least about 5 times greater than the dual layer graft 100, such as about 5.7 times greater. The added thickness may increase the handling properties of the ACA graft 100″. The added thickness may further give the ACA graft 100″ more biochemical complexity improve its protective barrier properties, and/or reduce its resorption rate.

The ACA graft 100″ may have a thickness of at least 50 micrometers, alternatively a thickness of at least 75 micrometers, alternatively a thickness of at least 100 micrometers, alternatively a thickness of at least 125 micrometers, alternatively a thickness of at least 150 micrometers, and alternatively a thickness of at least 200 micrometers. In some instance, the ACA graft 100″ may have a thickness in a range of from about 50 to about 200 micrometers, and alternatively from about 100 to about 150 micrometers. In some embodiments, as illustrated in Table 1, the ACA graft 100″ may have a thickness of 153.19±57.74, the full-thickness graft 100′ may have a thickness of 133.23±77.77, and the dual layer graft 100 may have a thickness of 26.97±16.89.

FIG. 3 is a stacked column graph illustrating the compositional analysis of the multi-layer grafts of FIGS. 2A-C based on proportional thickness. The compositional analysis was performed for 7 samples of each embodiment.

As further illustrated in Table 1 and FIG. 3, the dual layer graft 100 may comprise a substantial amount of amnion. For example, the dual layer graft 100 may have substantially 100% amnion including the first amnion 102 and the second amnion 202, with the chorion and the intermediate layers of the placental tissue removed.

The full-thickness graft 100′ may have substantially more chorion and intermediate layer than the dual layer graft 100. Thus, as indicated in Table 1, the full-thickness graft 100′ may constitute about 4% to about 12% amnion (e.g., about 10% amnion), about 11% to about 33% intermediate layer (e.g., about 15% intermediate layer), and from about 56% to about 86% chorion (e.g., about 75% chorion), inclusive, of the total thickness of the full-thickness graft 100′. In some embodiments, the full-thickness graft 100′ may be composed of approximately 71% chorion, 9% amnion, and 20% intermediate layer.

As further illustrated in FIG. 3, the ACA graft 100″ may have proportionally less amnion than the dual layer graft 100 (due to the retention of the chorion 106 and the intermediate layer 104) and more amnion than the full-thickness graft 100′ (due to the addition of the second amnion 202). Thus, the ACA graft 100″ may have proportionally less intermediate layer 104 and chorion 106 than the full-thickness graft 100′.

As illustrated, the first amnion 102 and the second amnion 202 may combine to constitute at least 5%, alternatively at least 10%, and alternatively at least 15% of an amnion, based on the thickness of the ACA graft 100″. In some embodiments, the first amnion 102 and the second amnion 202 of the ACA graft 100″ may have a combined thickness constituting from about 9% to about 29%, inclusive, of a total thickness of the ACA graft 100″. In some embodiments, the combined thickness of the first amnion 102 and the second amnion 202 may constitute from about 12% to about 22%, inclusive, of the total thickness of the ACA graft 100″.

As further illustrated, the chorion 106 may have a thickness constituting at least 20%, alternatively at least 25%, alternatively at least 30%, alternatively at least 35%, alternatively at least 40%, alternatively at least 45%, and alternatively at least 50%, based on the thickness of the ACA graft 100″. In some instances, the ACA graft 100″ may have chorion from about 38% to about 81%, inclusive, of the total thickness of the multi-layer graft. In some embodiments, the thickness of the chorion 106 may constitute about 60% to about 75%, inclusive, of the total thickness of the ACA graft 100″.

As further illustrated, the intermediate layer 104 may have a thickness constituting at least 2%, alternatively at least 5%, alternatively at least 10%, and alternatively at least 15% of an intermediate layer, based on the thickness of the ACA graft 100″. In some embodiments, the intermediate layer may constitute from about 7% to about 36%, inclusive, of the total thickness of the ACA graft 100″. In some embodiments, the intermediate layer may constitute from about 8% to about 22%, inclusive, of the total thickness of the ACA graft 100″.

In some embodiments, the ACA graft 100″ may consist of approximately 67% chorion, 17% amnion, and 15% intermediate layer.

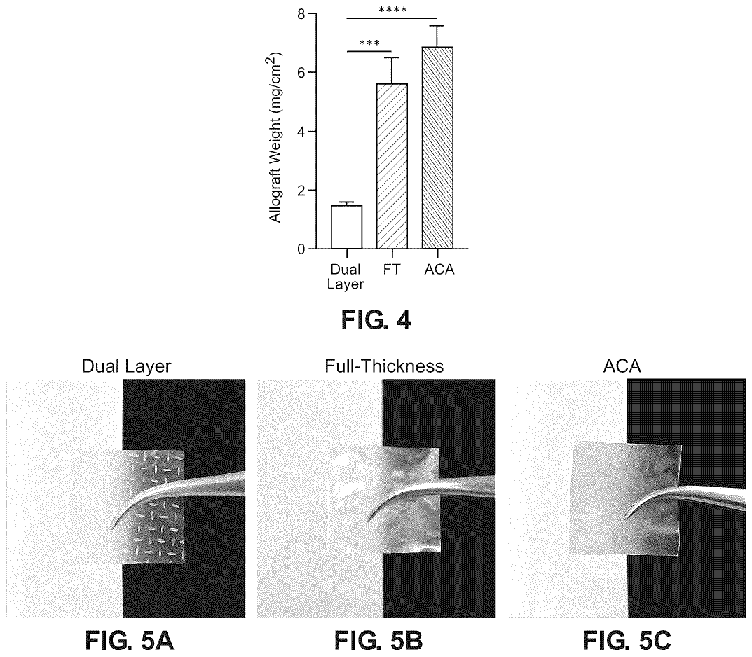

FIG. 4 is a bar graph illustrating the weight of the multi-layer grafts of FIGS. 2A-C. The mean weight of 8 samples per allograft technology were normalized to the surface area.

As illustrated, the weight of the full-thickness graft 100′ may be significantly greater than the dual layer graft 100 due to the addition of the chorion 106 and intermediate layer 104. The weight of the ACA graft 100″ may be greater than the dual layer 10 due to the intermediate layer 104 and the chorion 106. The weight of the ACA graft 100″ may be greater than the full-thickness graft 100′ due to the addition of the second amnion 202.

The dual layer may weigh less than 2 mg/cm² due to the removal of the chorion and intermediate layer, and the total weight of the full-thickness graft 100′ may be at least 4 mg/cm². In some embodiments, the weight of the ACA graft 100″ is at least 4 mg/cm², alternately at least 5 mg/cm², alternatively at least 6 mg/cm², and alternatively at least 6.5 mg/cm². The additional weight of the ACA graft 100″ contributes to the ease of handling of the ACA graft 100″.

FIG. 5A is an image of the dual layer graft 10, FIG. 5B is an image of the full-thickness graft 100′, and FIG. 5C an image of the ACA graft 100″. As illustrated in FIG. 5A, the dual layer graft 100 may be substantially transparent due to the transparency of each of the first amnion 102 and the second amnion 202. As illustrated in FIG. 5B, the full-thickness graft 100′ may be more opaque than the dual layer graft 100 due to the opacity of the chorion 106 and the intermediate layer 104. As illustrated in FIG. 5C, the ACA graft 100″ may be more opaque than the full-thickness graft 100′ due to the addition of the second amnion 202. The opacity of the ACA graft 100″ may provide advantages in a number of applications. For example, the ACA graft 100″ may provide improved visibility against lighter surfaces that allows the user to ensure proper placement. On the other hand, increased transparency of the full thickness graft 100′ may provide improved visibility of the wound space to better monitor the progression of healing.

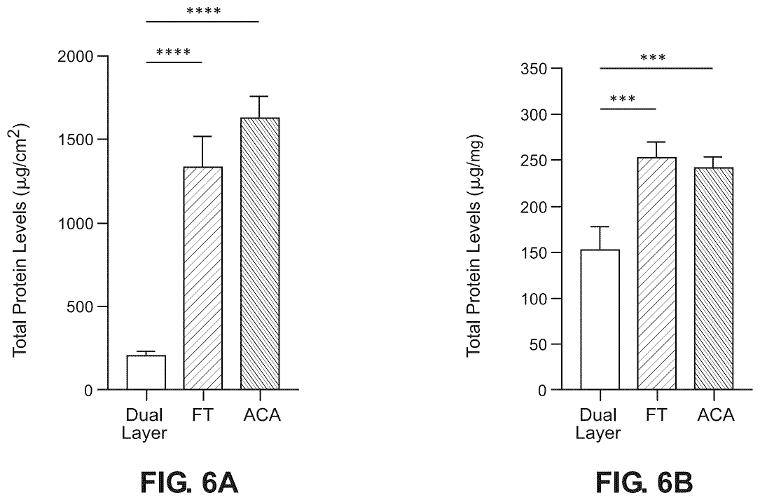

FIG. 6A is a bar graph illustrating the total protein levels of the multi-layer grafts, normalized by surface area. FIG. 6B is a bar graph illustrating the total protein levels of the multi-layer grafts, normalized by weight. Protein content was measured in the placental multi-layer grafts to investigate whether variations in layer composition influence overall protein levels. To ensure consistent comparison between the multi-layer grafts, total protein was normalized to both surface area and weight. Both the full-thickness graft 100′ and the ACA graft 100″ may exhibited significantly higher total protein levels compared to the dual layer graft 10, when normalizing by surface area or weight. Error bars represent the standard error mean (SEM). Statistical analysis was performed using one-way ANOVA with Tukey post-hoc test. ***, p<0.001; ****, p<0.0001.

As illustrated, the full-thickness graft 100′ demonstrated a 6-fold increase in total protein content relative to the dual layer graft 10. As further illustrated the ACA graft 100″ showed a 7-fold increase in total protein content relative to the dual layer graft 100. These differences reflect the inclusion of the intermediate layer 104 and the chorion 106 in the full-thickness graft 100′ and the ACA graft 100″. When normalized to overall multi-layer graft weight, the full-thickness graft 100′ and the ACA graft 100″ still exhibited significantly higher levels of total protein concentration, containing approximately 1.6 times more total protein compared to Dual Layer (FIG. 3B). These findings underscore the impact of layer composition and thickness on the total protein content of placental-derived multi-layer grafts.

The increased protein levels may be attributed to the full-thickness graft 100′ and ACA graft 100″ having more placental layers than the dual layer graft 100. As further illustrated in FIG. 6A, the ACA graft 100″ may have a higher protein concentration than the full-thickness graft 100′ when normalized by the surface area due to the inclusion of the second amnion 202.

In some embodiments, the total protein level of the ACA graft 100″ is at least 1200 μg/cm², alternatively at least 1400 μg/cm², and alternatively at least 1500 μg/cm² normalized by surface area.

In some embodiments, the total protein level of the ACA graft 100″ is at least 175 μg/mg, alternatively at least 200 μg/mg, and alternatively at least 225 μg/mg normalized by weight.

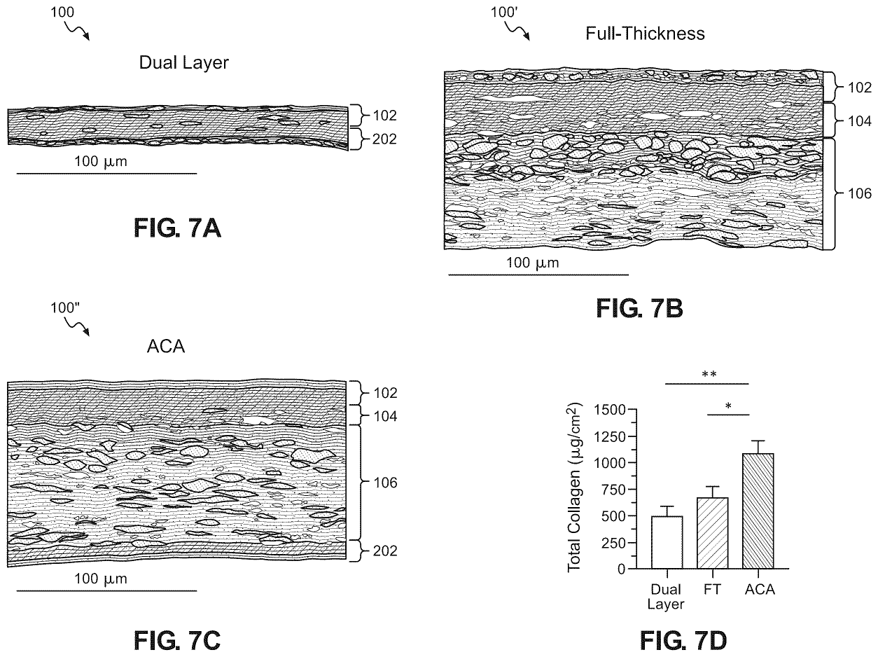

FIG. 7A illustrates the dual layer graft 10, FIG. 7B illustrates the full-thickness graft 100′, and FIG. 7C illustrates the ACA graft 100″, each stained for collagen with denser collagen areas indicated with hashmarks. Images were taken at 40× magnification to highlight the differences between multi-layer grafts. FIG. 7D is a bar graph illustrating the collagen levels of FIGS. 7A-C based on the surface area of the graft. Levels of total collagen are represented as the average of at least 7 donors per technology. *, p<0.05; **, p<0.01.

Collagen constitutes a critical group of proteins in the ECM of the placental tissue. Collagen provides structural integrity to the placental tissue supporting the handling and durability of the placental tissue. Collagen is further essential for applications involving a physical barrier, for example by reducing the risk of external contamination while supporting local tissue integrity.

As illustrated, the amnion 102, 202 is highly dense with collagen, specifically in the basement membrane and the compact layer. Thus, the dual layer graft 100 has high density of collagen throughout the first amnion 102 and the second amnion 202. The full-thickness graft 100′ has collagen throughout but the collagen varies across the layers 102-106 corresponding to its more complex structural architecture. The amnion 102 of the full-thickness graft 100′ has a high density of collagen (similar to the dual layer graft 10), followed by the intermediate layer which is similarly dense with collagen. The chorion 106 still has collagen but has a lower density. Similarly, the ACA graft 100″ has a high density of collagen through the first amnion 102, the intermediate layer 104, and the second amnion 202, with additional collagen provided by the chorion 106.

Thus, as illustrated in FIG. 7D, the ACA graft 100″ may have significantly more collagen than the dual layer graft 100 and the full thickness graft 100′ based on surface area. The ACA graft 100″ may have significantly more collagen than the dual layer graft 100 due to the contribution of collagen from the intermediate layer 104 and the chorion 106. The ACA graft 100″ may have significantly more collagen than the full-thickness graft 100′ due to the high concentration of collagen in the second amnion 202.

In some instances, the ACA graft 100″ has collagen in an amount of at least 500 μg/cm², alternatively at least 1250 μg/cm², alternatively at least 1500 μg/cm², and alternatively at least 1750 μg/cm² normalized by surface area. In some instances, the ACA graft 100″ has collagen in an amount ranging of from 500 μg/cm² to 2000 μg/cm², alternatively from 750 μg/cm² to 1500 μg/cm², alternatively from 750 μg/cm² to 1250 μg/cm², and alternatively from 950 μg/cm² to 1250 μg/cm² normalized by surface area. In some instances, the ACA graft 100″ has collagen in an amount of at least 50 μg/mg, alternatively at least 100 μg/mg, and alternatively at least 150 μg/mg normalized by weight.

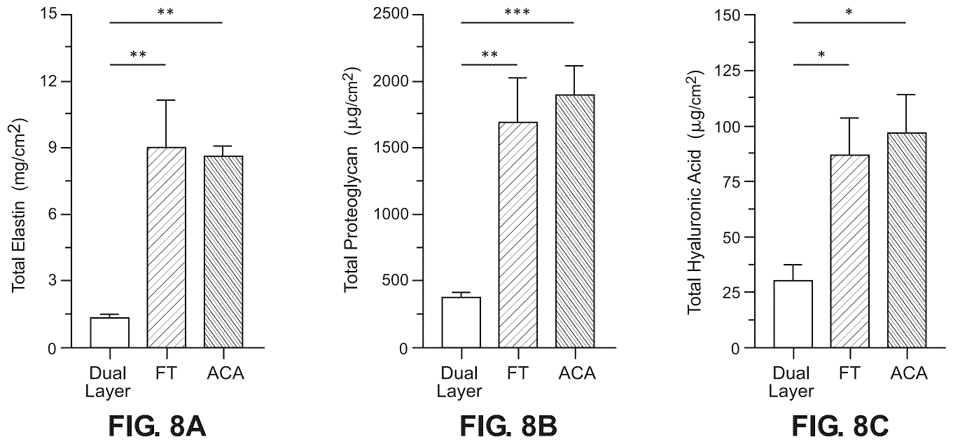

FIG. 8A is a graph illustrating the total elastin levels, FIG. 8B is a graph illustrating the total proteoglycan levels, and FIG. 8C is a graph illustrating the total hyaluronic acid levels for each of the multi-layer grafts of FIGS. 2A-C. The concentrations are quantified in at least 8 donors per product. Error bars represent the standard error mean (SEM). Statistical analysis was performed using one-way ANOVA with Tukey post-hoc test. *, p<0.05; **, p<0.01; ***, p<0.001.

Elastin and proteoglycans have been shown to support the formation of a protective barrier in the placental tissue. Elastin has been further suggested to be an important protein to increasing flexibility of tissue coverings. Proteoglycans and HA are known to facilitate hydration and structural organization in various tissues. Hyaluronic acid, elastin, and proteoglycans further aid in tissue remodeling, while elastin further contributes to the structural integrity of the placental tissue.

FIGS. 8A-C illustrates that the full-thickness graft 100′ and the ACA graft 100″ retains structural complexity of various proteins. For example, FIG. 8 illustrates that the full-thickness graft 100′ and the ACA graft 100″ demonstrate high levels of elastin, which may be attributable to the high level of elastin that has been reported for the chorion 106 and may contribute to the protective barrier function. FIG. 8B-C illustrates that the full-thickness graft 100′ and the ACA graft 100″ demonstrate high levels of proteoglycans and hyaluronic acid that contributes to the hydration and structural organization of the placental tissue. FIG. 8B-C further illustrates that the addition of the second amnion 202 in the ACA graft 100″ may increase the concentration of proteoglycans and hyaluronic acid, in comparison to the full-thickness graft 100′.

In some instances, the ACA graft 100″ may have elastin in an amount of at least 4 mg/cm², alternatively at least 5 mg/cm², alternatively at least 6 mg/cm², alternatively at least 7 mg/cm², and alternatively at least 8 mg/cm² normalized by surface area. In some instances, elastin is present in the ACA graft 100″ in a range of from 5 mg/cm² to 14 mg/cm², alternatively from about 7 mg/cm² to about 10 mg/cm², alternative from about 7 mg/cm² to about 9 mg/cm², alternatively from about 8 mg/cm² to about mg/cm² normalized by surface area.

In some instances, the ACA graft 100″ may have proteoglycans in an amount of at least 750 μg/cm², alternatively at least 1250 μg/cm², alternatively at least 1500 μg/cm², and alternatively at least 1750 μg/cm² normalized by surface area. In some instances, the proteoglycan is present in the ACA graft 100″ an amount ranging of 750 μg/cm² to about 3250 μg/cm², alternatively from 1000 μg/cm² to 3000 μg/cm², alternatively from 1250 μg/cm² to about 2250 μg/cm², alternatively from about 1500 μg/cm² to about 2250 μg/cm², alternatively from about 1500 μg/cm² to about 2000 μg/cm², and alternatively from about 1750 μg/cm² to about 2000 μg/cm² normalized by surface area.

In some instances, the ACA graft 100″ may have hyaluronic acid in an amount of at least 20 μg/cm², alternatively at least 50 μg/cm², alternatively at least 75 μg/cm², alternatively at least 85 μg/cm², alternatively at least 90 μg/cm², and alternatively at least 95 μg/cm² normalized by surface area. In some instances the hyaluronic acid is present in the ACA graft 100″ in an amount of from 75 μg/cm² to about 125 μg/cm², alternatively from about 75 μg/cm² to about 115 μg/cm², and alternatively from about 85 μg/cm² to about 115 μg/cm² normalized by surface area.

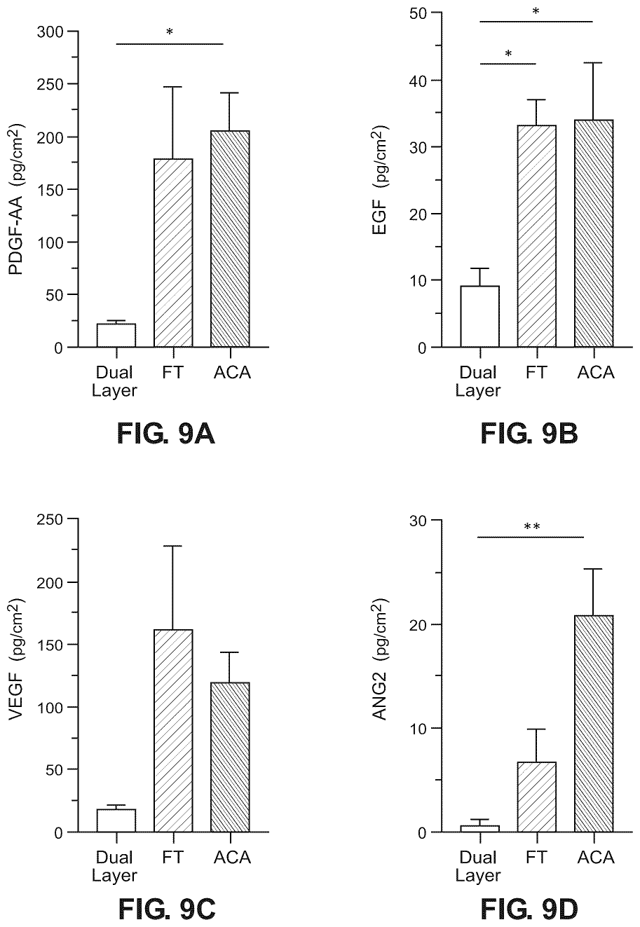

FIG. 9A is a graph illustrating a concentration of platelet-derived growth factor-AA (PDGF-AA) levels, FIG. 9B is a graph illustrating a concentration of epidermal growth factor (EGF) levels, FIG. 9C is a graph illustrating a concentration of vascular endothelial growth factor (VEGF) levels, and FIG. 9D is a graph illustrating a concentration of angiopoietin-2 (ANG2) levels, for each of the multi-layer grafts of FIGS. 2A-C. The concentrations were quantified in 8 donors per allograft/CAMP technology. Error bars represent the standard error of means (SEM). *, p<0.05, **, p<0.01.

Table 2 provides exemplary levels of growth factors.

TABLE 2

| Growth | | | |
|---|---|---|---|
| Factor Levels | | | |
| (pg/cm2) | Dual Layer | Full-Thickness | ACA |
| ANG-2 | 0.61 ± 1.36 | 6.73 ± 7.75 | 20.88 ± 11.91 |
| EGF | 9.12 ± 6.95 | 33.05 ± 10.85 | 33.91 ± 24.25 |
| PDGF | 22.30 ± 8.50 | 178.91 ± 193.71 | 204.74 ± 97.03 |
| VEGF | 18.46 ± 6.70 | 161.65 ± 188.69 | 199.10 ± 59.74 |

Values are represented in mean ± standard deviation (SD).

FIGS. 9A-D and Table 2 illustrate that the full-thickness graft 100′ and the ACA graft 100″ retains high levels of intrinsic growth factors relative to the dual layer graft 100. This aligns with findings that the intermediate layer 104 is a major source of these growth factors. As further illustrated, the ACA graft 100″ may have higher levels of ANG-2, PDGF, and VEGF than the full-thickness graft 100′ due to the addition of the second amnion 202 in the ACA graft 100″. The retained growth factors may be used to support advanced uses of CAMPs. For instance, the retained EGF may promote cell proliferation, differentiation, and cell survival. The retained VEGF may promote endothelial cell function. The retained PDGF may promote with cell recruitment. The retained ANG-2 may promote angiogenic regulation. The increased presence of these molecules in the full-thickness graft 100′ and the ACA graft 100″ suggests a more complex biochemical environment which supports future research into potential advancements.

In some instances, the ACA graft 100″ has PDGF-AA in an amount of at least 150 pg/cm², alternatively at least 175 pg/cm², alternatively at least 190 pg/cm², and alternatively at least 200 pg/cm² normalized by surface area. In some instances, the ACA graft 100″ has PDGF-AA in an amount of from about 125 pg/cm² to about 225 pg/cm², alternatively from about 150 pg/cm² to about 225 pg/cm², alternatively from about 150 pg/cm² to about 200 pg/cm², and alternatively from about 175 pg/cm² to about 200 pg/cm² normalized by surface area.

In some instances, the ACA graft 100″ has EGF in an amount of at least 25 pg/cm², alternatively at least 28 pg/cm², and alternatively at least 30 pg/cm² normalized by surface area. In some instances, the ACA graft 100″ has EGF in an amount of from about 20 pg/cm² to about 35 pg/cm², alternatively from about 25 pg/cm² to about 35 pg/cm², and alternatively from about 28 pg/cm² to about 35 pg/cm² normalized by surface area.

In some instances, the ACA graft 100″ has VEGF in an amount of at least 75 pg/cm², alternatively at least 80 pg/cm², alternatively at least 90 pg/cm², and alternatively at least 100 pg/cm² normalized by surface area. In some instances, the ACA graft 100″ has VEGF in an amount of from about 75 pg/cm² to about 125 pg/cm², alternatively from about 75 pg/cm² to about 100 pg/cm², and alternatively from about 85 pg/cm² to about 100 pg/cm² normalized by surface area.

In some instances, the ACA graft 100″ has ANG2 in an amount of at least 10 pg/cm², alternatively at least 15 pg/cm², and alternatively at least 18 pg/cm² normalized by surface area. In some instances, the ACA graft 100″ has ANG2 in an amount of from 10 pg/cm² to about 25 pg/cm², alternatively from about 15 pg/cm² to about 25 pg/cm², and alternatively from about 15 pg/cm² to about 20 pg/cm² normalized by surface area.

The disclosed embodiments demonstrate that incorporating the intermediate layer 104, and in the case of ACA, an additional amnion 202, may result in higher levels of total protein, collagen, elastin, proteoglycans, HA, and key growth factors in placental-derived multi-layer grafts, categorized as CAMPs. The multi-layer designs (e.g., the full-thickness graft 100′ and the ACA graft 100″) exhibit a broader biochemical profile than more simple constructs such as the dual layer graft 100.

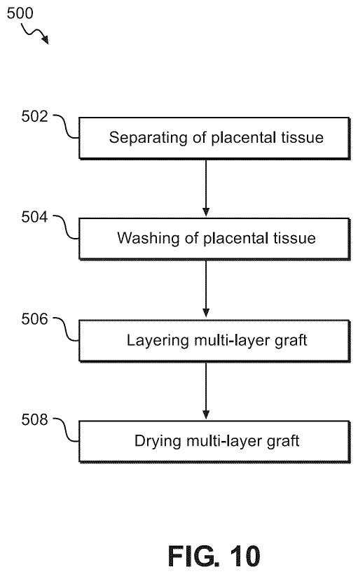

FIG. 10 illustrates a method 500 of forming a multi-layer graft, such as the full-thickness graft 100′ and/or ACA graft 100″. The method 500 may include processing techniques characterized as minimally manipulative to retain natural properties of the tissue.

The method 500 may include receiving a placental membrane 10 from a health donor. In some embodiments, the donor is human. The placental membrane 10 may be acquired at the time of delivery from healthy birth mothers, whom have been screened and tested in accordance with U.S. Food and Drug Administration (FDA) regulations and American Association of Tissue Banks (AATB) standards to ensure the safety and quality of the tissue.

In step 502, the method 500 may include separating placental tissue from the placental membrane 10. Step 502 may include separating the placental membrane 10 from the placenta 16 with blunt dissection. The placental membrane 10 may then be rinsed with buffered salt and detergent solutions. The method 500 may further include separating one or more placental tissues from the placental membrane 10. For the dual layer graft 10, the amnion 102 may be delaminated from the chorion 104. The dual layer graft 100 may be formed by a single placental sheet of amnion 102 folded back-to-back. For the full-thickness graft 100′ and the ACA graft 100″, a first placental tissue in a full thickness of the placental membrane 10 may be separated. The first placental tissue may include the amnion 102, the intermediate layer 104, and the chorion 106. The method 500 may include retaining the full-thickness of the first placental tissue substantially intact, at least partially preserving the first amnion 102, the intermediate layer 104, and the chorion 106 in its natural state.

In some embodiments, the first placental tissue may become at least partially separated during extracting from the donor. Thus, step 502 may include reconstituting the first placental tissue after extraction. Reconstituting may include stretching and extending the amnion 102 over the chorion 106 (e.g., with at least part of the intermediate layer 104 therebetween) to create a smooth surface of the placental tissue. The intermediate layer 104 may be used to adhere the amnion 102 and the chorion 104.

In embodiments of the ACA graft 100″, a second placental tissue may be extracted from the donor. The second placental tissue may include the second amnion 202. The second placental tissue may be derived at least partially from the placental globe 14 of the membrane 10. The placental globe 14 may facilitate handling due to natural separation of the amnion 202 from the chorion at that anatomical site. In some embodiments without complete natural separation, the second amnion 202 may be delaminated from the chorion. In some embodiments, at least a portion of the intermediate layer may be retained on the second amnion 202. For example, the entire intermediate layer of the second placental tissue may be retained for the proteins and/or growth factors. In some embodiments, at least a portion of the intermediate layer may be removed (intentionally or unintentionally) from the second amnion 202. For example, the intermediate layer may be removed from the second amnion 202 through scraping. The intermediate layer may be removed to expose the fibrous layer for attachment of the second amnion 202 to the chorion 106.

The first placental tissue and the second placental tissue may be derived from the placental membrane 10 from the same donor. For example, the second placental tissue may be at least partially derived from the placental globe 14 of the placental membrane 10 due to the natural separation of the amnion 202, and the first placental tissue may be at least partially derived from the portion 20 of the placental membrane excluding the placental globe 14 due the natural lamination of the amnion 102 and the chorion 106.

In step 504, the method 500 may include washing the placental tissue. The first placental tissue and the second placental tissue may be washed before or after forming the multi-layer graft. In some instances, the placental tissue(s) are washed on a shaker. In some instances, the shaker is moving at a speed in a range of from 50 RPM to 500 RPM, alternatively from 50 RPM to 250 RPM, and alternatively from 50 RPM to 150 RPM. In some instances, the placental tissue(s) are washed at least once in a salt solution. In some instances, the salt solution comprises at least one of sodium chloride, potassium chloride, magnesium chloride, sodium phosphate, potassium phosphate, magnesium phosphate, sodium acetate, potassium acetate, magnesium acetate, calcium chloride, calcium phosphate, or calcium acetate. In some instances, the placental tissue(s) are washed in the salt solution for at least 5 minutes, alternatively at least 10 minutes, and alternatively at least 15 minutes. In some instances, the placental tissue(s) are washed in at least one surfactant. In some instances, the surfactant is a non-ionic surfactant. In some instances, the surfactant is an anionic surfactant. In some instances, the surfactant is a cationic surfactant. In some instances, the surfactant is a detergent. In some instances, the surfactant is NP-40, Triton X-100, Tween 20, CHAPS, urea, sodium dodecyl sulfate (SDS), deoxycholate, cholate, sarkosyl, DDM, digitonin, or Tween 80. In some instances, the surfactant is a detergent. In some instances, the surfactant is a polyethylene glycol-based detergent. In some instances, the surfactant is diluted. In some instances, the surfactant is diluted to a 0.001-2.00% solution to be used. In some instances, the surfactant is diluted to a 0.01-2.00% solution to be used. In some instances, the surfactant is diluted to a 0.01-1.00% solution to be used. In some instances, the layers are washed in a surfactant for at least 5 minutes, alternatively at least 10 minutes, alternatively at least 15 minutes, and alternatively at least 20 minutes. In some instances, the layers are washed at least once in a saline solution. In some instances, the saline solution is phosphate buffer saline solution. In some instances, the saline solution is, for example, RIS-buffered saline, HEPES-buffered saline, standard saline citrate, Hank's balanced salt solution, Earle's balanced salt solution, Gey's balanced salt solution (GBSS). In some instances, the layers are washed in a saline solution for at least 5 minutes, alternatively at least 10 minutes, and alternatively at least 15 minutes. In some instances, the placental tissue(s) are washed in water for at least 5 minutes, alternatively at least 10 minutes, and alternatively at least 15 minutes.

In step 506, the method 500 may include layering the placental tissues to form the ACA graft 100″. In some instances, the second amnion 202 is spread over a surface. In some instances, the maternal side of the chorion 106 is placed on the stromal side of the second amnion 202, with the epithelial sides of the first amnion 102 and the second amnion 202 exposed. In some instances, the second amnion 202 comprises a second intermediate layer. In some instances, the chorion 106 is placed in direct contact with the second amnion 202. As discussed above, the adhesion of the layers may be formed by the intermediate layer of the second amnion 202 and/or an exposed portion of the fibrous layer of the second amnion 202. The intermediate layer of the second amnion 202 may form a sticky substance that adheres the layers and drying may stabilize the ACA graft 100″.

In some embodiments, the method 500 may include separating an amnion portion from the first placental tissue forming the first amnion 102, based on the second amnion 202 insufficiently covering the maternal side of the chorion 106. This may be useful when an insufficient length of the placental globe 14 is available. A chorion portion of the chorion 106 that was fused to the amnion portion may be separated from the chorion 106 (e.g., during the separation of the amnion portion) and discarded. The stromal side of the amnion portion from the first amnion 202 may be applied to the maternal side of the chorion 106 to sufficiently cover the chorion 106.

In step 508, the method 500 may include drying the multi-layered graft which may adhere and/or preserve the graft. The multi-layered graft may be dried at a high temperature ranging of from 50° F. to 350° F., alternatively from 150° F. to 300° F., alternatively of from 50° F. to 200° F., alternatively from 100° F. to 200° F., and alternatively from 200° F. to 300° F. The multi-layered graft may be dried at the high temperature in a range of from about 1 second to about 1 minute, alternative from about 1 second to about 30 seconds, and alternative from about 1 second to about 10 seconds. Additionally or alternatively, the multi-layered graft may be dried at a low temperature ranging of from 20° F. to 50° F., alternatively from 30° F. to 50° F. and alternatively from 30° F. to 45° F. The multi-layered graft may be dried at the low temperature in a range of from about 25 hours to about 50 hours, alternatively about 30 hours to about 50 hours, and alternatively about 40 to about 50 hours. The drying may dehydrate the one or more layers of the ACA graft and bond the layers together. In some embodiments, drying contributes to the shelf life of the ACA graft. In some embodiments, drying contributes to preservation as the removal of water is detrimental to the growth of microorganisms. In some embodiments, drying contributes to a multi-layer graft that is lighter and weighs less.

In step 510, the method 500 may include packaging and sterilizing the graft using gamma-irradiation to ensure microbial safety.

The resultant multi-layer graft may be used for various applications. The multi-layer graft may used for an ocular implant. In some instances, the multi-layer graft is used for wound covering such as, for example, but not limited to, partial- and full-thickness wounds, pressure ulcers, venous ulcers, diabetic ulcers, chronic vascular ulcers, tunneled/undermined wounds, surgical wounds (donor sites/grafts, post-Mohs surgery, post-laser surgery, podiatric, wound dehiscence), trauma wounds (abrasions, lacerations, second-degree burns and skin tears) draining wounds, chronic wounds, non-healing wounds, surgical burns, diabetic ulcers, or an acute wound. Additionally or alternatively, the multi-layer graft may be used as a tissue barrier, for example to prevent adhesion between two adjacent tissue.

The present disclosure also provides a kit of one or more of the multi-layer grafts 100, 100′, 100″. For example, the inventors recognize the importance of carefully selecting and processing placental membranes to optimize the composition and functionality of CAMPs for different clinical uses. In cases where a thicker covering with a broader biochemical profile is needed, the full-thickness graft 100′ and/or the ACA graft 100″ might be favored. In cases where a thinner covering may be needed, the dual layer graft 100 may be favored. Furthermore, the flexibility in design reflects how processing decisions, whether to omit or retain certain placental layers, are likely to directly implicate the allograft's biological properties. Thus, the present disclosure includes a kit including one or more of the dual layer graft 100, the full-thickness graft 100′, and/or the ACA graft 100′″. Providing a plurality of different multilayer grafts 100, 100′, 100″ may allow the user to select and choose between grafts for the specific applications and/or treatment site. For example, the plurality of different multilayer grafts 100, 100′, 100″ may be packaged together in a box or outer container and/or individually packaged inside of the box or outer container.

1. A multi-layer graft comprising:
a first amnion having a first stromal side and a first epithelial side;
a chorion; and
a second amnion having a second stromal side and a second epithelial side,
wherein the chorion is positioned between the first stromal side and the second stromal side, and the first epithelial side and the second epithelial side are exposed.

2. The multi-layer graft of claim 1, further comprising an intermediate layer between the first amnion and the chorion.

3. The multi-layer graft of claim 2, wherein the first amnion, the intermediate layer, and the chorion are derived from a full thickness of a placental tissue.

4. The multi-layer graft of claim 1, wherein the first amnion is at least partially derived from a first portion of a placental membrane, and the second amnion is at least partially derived from a second portion of the placental membrane.

5. The multi-layer graft of claim 4, wherein the second portion is a placental globe, and the first portion is a portion of the placental membrane excluding the placental globe.

6. The multi-layer graft of claim 1, wherein the second amnion is in direct contact with the chorion.

7. The multi-layer graft of claim 1, wherein the first epithelial side of the first amnion has a first layer of epithelial cells, and the second epithelial side of the second amnion has a second layer of epithelial cells.

8. The multi-layer graft of claim 1, wherein the multi-layer graft has a thickness of at least about 100 microns.

9. The multi-layer graft of claim 1, wherein the chorion has a thickness constituting from about 38% to about 81%, inclusive, of a total thickness of the multi-layer graft.

10. The multi-layer graft of claim 9, wherein the thickness of the chorion constitutes about 60% to about 75%, inclusive, of the total thickness of the multi-layer graft.

11. The multi-layer graft of claim 1, wherein the first amnion and the second amnion have a combined thickness constituting from about 9% to about 29%, inclusive, of a total thickness of the multi-layer graft.

12. The multi-layer graft of claim 11, wherein the combined thickness of the first amnion and the second amnion constitutes from about 12% to about 22%, inclusive, of the total thickness of the multi-layer graft.

13. The multi-layer graft of claim 2, wherein the intermediate layer has a thickness constituting from about 7% to about 36%, inclusive, of a total thickness of the multi-layer graft.

14. The multi-layer graft of claim 13, wherein the thickness of the intermediate layer constitutes from about 8% to about 18%, inclusive, of the total thickness of the multi-layer graft.

15. The multi-layer graft of claim 1, wherein the multi-layer graft has collagen in a concentration of from about 500 μg/cm² to about 1750 μg/cm², inclusive.

16. The multi-layer graft of claim 1, wherein the multi-layer graft has elastin in a concentration of from about 7 mg/cm² to about 14 mg/cm², inclusive.

17. The multi-layer graft of claim 1, wherein the multi-layer graft has proteoglycans in a concentration of from about 750 μg/cm² to about 3250 μg/cm², inclusive.

18. The multi-layer graft of claim 1, wherein the multi-layer graft has hyaluronic acid in a concentration of from about 25 μg/cm² to about 175 μg/cm², inclusive.

19. The multi-layer graft of claim 1, wherein the first amnion, the chorion, and the second amnion are adhered together.

20. A method comprising:
receiving a first amnion and a chorion derived from a first placental tissue;
receiving a second amnion derived from a second placental tissue; and
applying a stromal side of the second amnion to a maternal side of the chorion to form a multi-layer graft with a first epithelial side of the first amnion and a second epithelial side of the second amnion exposed.

21. The method of claim 20, further comprising receiving an intermediate layer from the first placental tissue.

22. The method of claim 21, wherein the first amnion, the intermediate layer, and the chorion are derived from a full thickness of the first placental tissue.

23. The method of claim 20, wherein the first placental tissue is at least partially derived from a first portion of a placental membrane, and the second placental tissue is at least partially derived from a second portion of the placental membrane.

24. The method of claim 23, wherein the second portion is a placental globe, and the first portion is a portion of the placental membrane excluding the placental globe.

25. The method of claim 20, further comprising:
separating an amnion portion from the first amnion, based on the second amnion insufficiently covering the maternal side of the chorion; and
applying a stromal side of the amnion portion to the maternal side of the chorion.

26. The method of claim 25, further comprising:
separating a chorion portion from the chorion; and
discarding the chorion portion.
